(19) United States
(12) Patent Application Publication
(10) Pub. No.: US 2007/0005026 A1
(43) Pub. Date: Jan. 4, 2007

(54) METHOD AND MATERIAL FOR REDUCING BIOFILM

(76) Inventors: Michael Basara, Hugo, MN (US); James Bracke, Vadnais Heights, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/425,641

(22) Filed: Jun. 21, 2006

Related U.S. Application Data
(60) Provisional application No. 60/692,368, filed on Jun. 21, 2005.

(51) Int. Cl. A61M 35/00

(52) U.S. Cl. 604/289

(57) ABSTRACT

The present invention includes a method for removing a biofilm from a surface, comprising exposing the biofilm to a formulation comprising phenolsulfonic acid guaiacolsulfonic acid, sulfosalicylic acid, citric acid, and ammonium phenolsulfonate for at least about fifteen seconds.

### RELATED APPLICATION

This application claims the benefit of U.S. Provisional Application Ser. No. 60/692,368 filed Jun. 21, 2005, which application is incorporated herein by reference.

### FIELD

Embodiments of the invention described herein relate to a system for reducing biofilms, a method for making the system and a method for using the system to reduce biofilms.

### BACKGROUND

The term, “biofilm” as used herein refers to a material which naturally develops when microbes attach to a support that is made of a material including but not limited to stone, metal, plastic, glass and wood. “Biofilm” also refers to filamentous and non-filamentous bacteria that produce an extracellular polysaccharide and proteinaceous material that act as a natural glue to immobilize the cells. In nature, nonfilament-forming microorganisms stick to the biofilm surface, locating within an area of the biofilm that provides an optimal growth environment with respect to pH, eH, dissolved oxygen, and nutrients. Since nutrients tend to concentrate on solid surfaces, including porous surfaces and wet, dry surfaces, a microorganism saves energy through cell adhesion to a solid surface rather than by growing unattached.

Microbes are capable of attachment to almost any surface submerged in an aqueous environment—a phenomenon known as microbial adhesion. Colonization and proliferation of the microbes on a surface forms a biofilm.

Adhesion of microbes on a surface is involved in diseases of humans and animals, in dental plaque formation, in industrial processes, in fouling of man-made surfaces, in syntrophic and other community interactions between microorganisms, and in the activity and survival of microorganisms in natural habitats.

Poloxamer 407 has been shown to have an anti-adhesive effect on bacterial adherence to polymethylmethacrylate and enhanced the susceptibility of bacteria to antibiotics. Condensed tannins and methylcellulose were found to prevent microbial attachment and subsequent digestion of cellulose.

Some investigators have attempted to remove adherent bacteria through use of enzymes, which degrade their bridging polymers. An example is the application of glucan hydrolases, which attack the extracellular glucans, which promote accumulation of Streptococcus mutans cells on teeth.

Some proteins have also been found to reduce biofilm formation. Adhesion of a marine Pseudomonas to polystyrene decreased due to the presence of BSA, gelatin, fibrinogen, protamine and pepsin. When free proteins were present during the attachment, the strongest influence on adhesion was observed, presumably due to protein adsorption on both, the bacterial and polystyrene surface. Pretreatment of the polystyrene surface with proteins also led to a reduction of the adhesion (except pretreatment with protamine), whereas the pretreatment of bacteria resulted in decreased adhesion for BSA-treated cells only. Probably, the adsorption of proteins had a greater influence on the hydrophobicity of the substrate surface than on the surface of bacteria.

In addition, there have been several patents claiming methods for inhibiting the microbial adhesion on surfaces. Wright et al. U.S. Pat. No. 5,512,186 proposed use of combination of alkylsulfosuccinate surfactant with alkyl chain length from 5 to 13 carbon atoms and polyoxyethylene- polyoxypropylene block copolymer surfactant. Donald et al. used ethoxylated nonionic surfactant which is a block copolymer of repeating ethylene oxide and repeating propylene oxide units for inhibiting the microbial colonization of a hydrophobic surface.

### DESCRIPTION OF THE DRAWINGS

FIG. 1A is a schematic view of a prior art formulation that includes sulfuric acid, Beechwood creosote and purified water.

FIG. 1B is a schematic view of the formulation used in the system described herein and its uses in treating conditions of skin and the oral cavity.

FIG. 2 is a formula view of chemicals in the system described herein.

FIG. 3 is a perspective view of device component embodiments of the system.

FIG. 4A illustrates scanning electron micrograph images showing a bacterial lawn prior to treatment with a system embodiment of the invention.

FIG. 4B illustrates scanning electron micrograph images showing that about 80% of a bacterial biofilm was removed after fifteen second of exposure with a formulation embodiment described herein, designated in the Figure as “Debacterol.”

FIG. 5 illustrates scanning electron micrograph images showing that about 85% of a bacterial biofilm was removed after fifteen seconds of exposure with a formulation described herein, referred to as “HybenX.”

FIG. 6 illustrates a scanning electron micrograph image showing a control bacterial lawn prior to treatment with HybenX.

### SUMMARY

Embodiments of the invention include a method for removing a biofilm from a surface. The method includes exposing the biofilm to a formulation comprising sulfonic acid derivatives of hydroxybenzenes and hydroxymethoxybenzene for at least about fifteen seconds.

Another embodiment includes a system for removing a biofilm from a surface. The system includes a formulation comprising; sulfonic acid derivatives of hydroxybenzenes and hydroxymethoxybenzene and an applicator for applying the formulation to a biofilm for at least about 15 seconds.

Another embodiment includes a system for making a formulation for removing a specific type of biofilm from a surface comprising: receptacles for containing two or more ingredients of phenolsulfonic acid, guaiacolsulfonic acid, sulfosalicylic acid, ammonium phenolsulfonate, potassium guaiacolsulfonate; and a mechanism calibrated to mix the ingredients together to make a formulation for treating the specific type of biofilm.

One other embodiment includes a method for removing biofilm from seeds comprising exposing the seeds to a formulation comprising sulfonic acid derivatives of hydroxybenzenes and hydroxymethoxybenzene.

### DETAILED DESCRIPTION

Although detailed embodiments of the invention are disclosed herein, it is to be understood that the disclosed embodiments are merely exemplary of the invention that may be embodied in various and alternative forms. Specific structural and functional details disclosed herein are not to be interpreted as limiting, but merely as a basis for teaching one skilled in the art to variously employ the biofilm removal embodiments. Throughout the drawings, like elements are given like numerals.

Referred to herein are trade names for materials including, but not limited to, polymers and optional components. The inventors herein do not intend to be limited by materials described and referenced by a certain trade name. Equivalent materials (e.g., those obtained from a different source under a different name or catalog (reference) number to those referenced by trade name may be substituted and utilized in the methods described and claimed herein. All percentages and ratios are calculated by weight unless otherwise indicated. All percentages are calculated based on the total composition unless otherwise indicated. All component or composition concentrations are in reference to the active level of that component or composition, and are exclusive of impurities, for example, residual solvents or by-products, which may be present in commercially available sources.

In its method and system aspects, embodiments of the invention described herein include a method and system for removing at least about 80% of a biofilm from a surface within a period of time from substantially instantaneously to about 15 seconds. The method includes obtaining a formulation that includes sulfonic acid derivatives of hydroxybenzenes and hydroxymethoxybenzene. In one embodiment, the formulation includes a quantity of four isomers of phenolsulfonic acid, seven isomers of guaiacolsulfonic acid, and for some embodiments, acid and water. The specific isomer concentrations and concentration ratios are preselected by selecting specific reaction parameters such as time, temperature, and concentration of reactants that produce the preselected isomer concentration profile. The formulation is applied to a biofilm in a manner that covers the biofilm for about 15 seconds.

In another embodiment, the phenolsulfonic acid in a formulation for removing the biofilm is further treated with a hydroxide such as ammonium hydroxide to make ammonium phenolsulfonate before application to a biofilm. The guaiacolsulfonic acid is treated with a hydroxide such as potassium or zinc hydroxide to make potassium guaiacolsulfonate.

The system embodiments of the invention include formulations that include the ingredients phenolsulfonic acid, guaiacolsulfonic acid, sulfosalicylic acid, ammonium phenolsulfonate, potassium guaiacolsulfonate, water and sulfuric acid which is National Formulary (NF). At least one embodiment of the system also includes a mechanism calibrated to mix preselected amounts of ingredients together to make a formulation for treating a specific type of biofilm.

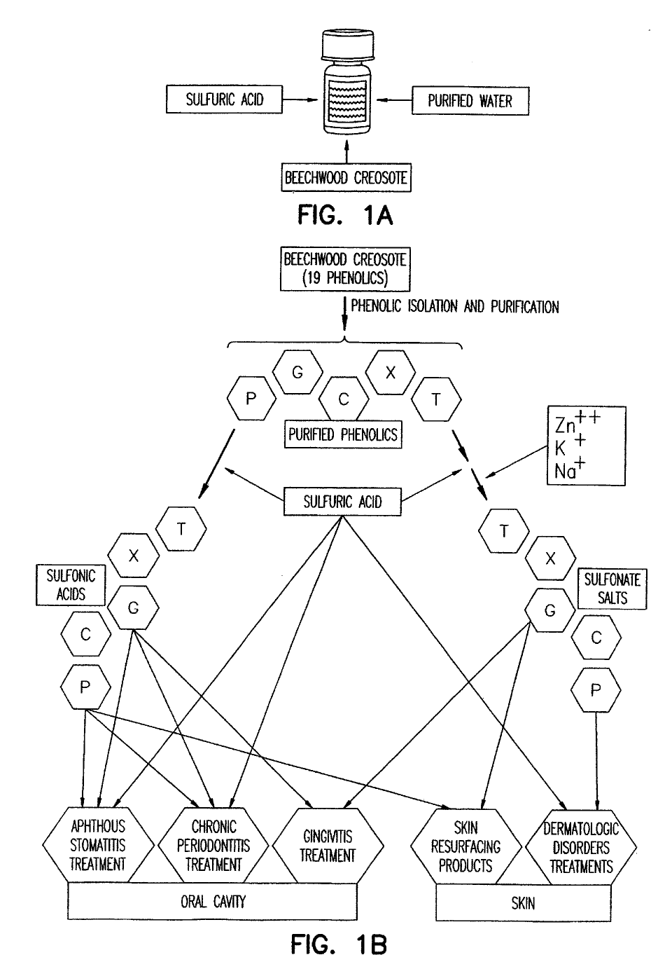

In its system aspect, embodiments of the invention include a formulation for treating bacterial biofilms that includes phenolsulfonic acid, sulfosalicylic acid, guaiacolsulfonic acid, and, for some embodiments, ammonium phenolsulfonate, sulfuric acid and water. In one embodiment, the formulation includes phenolic compounds that are isolated and purified, as is shown in FIG. 1B.

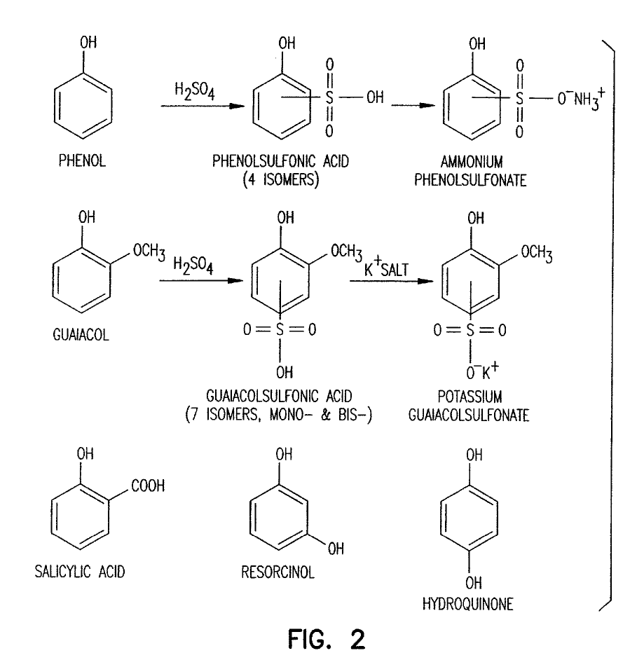

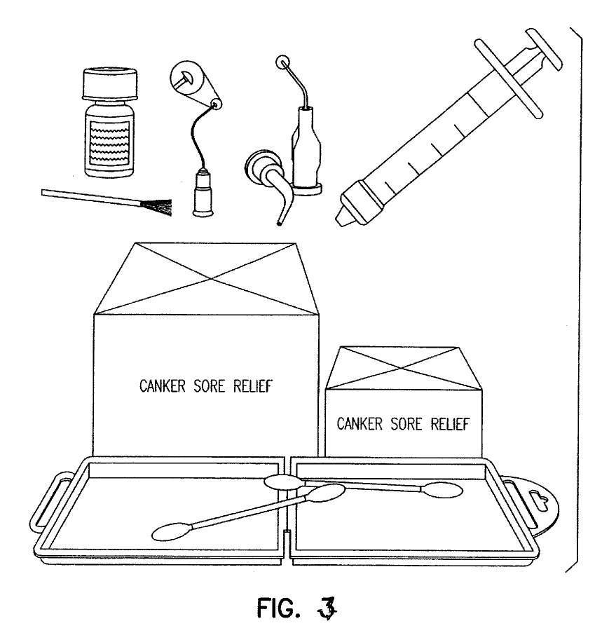

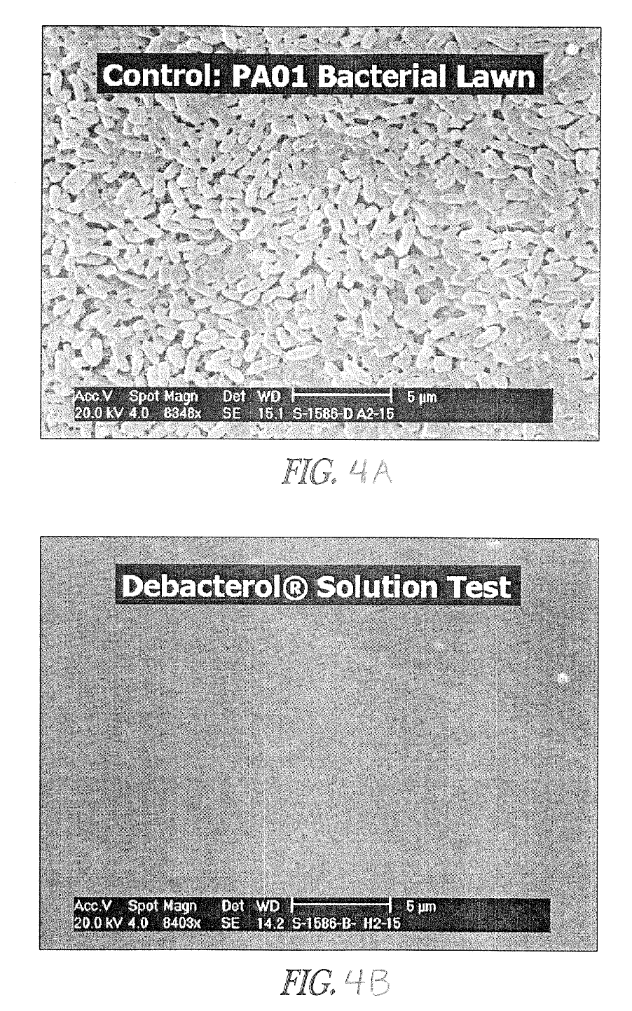

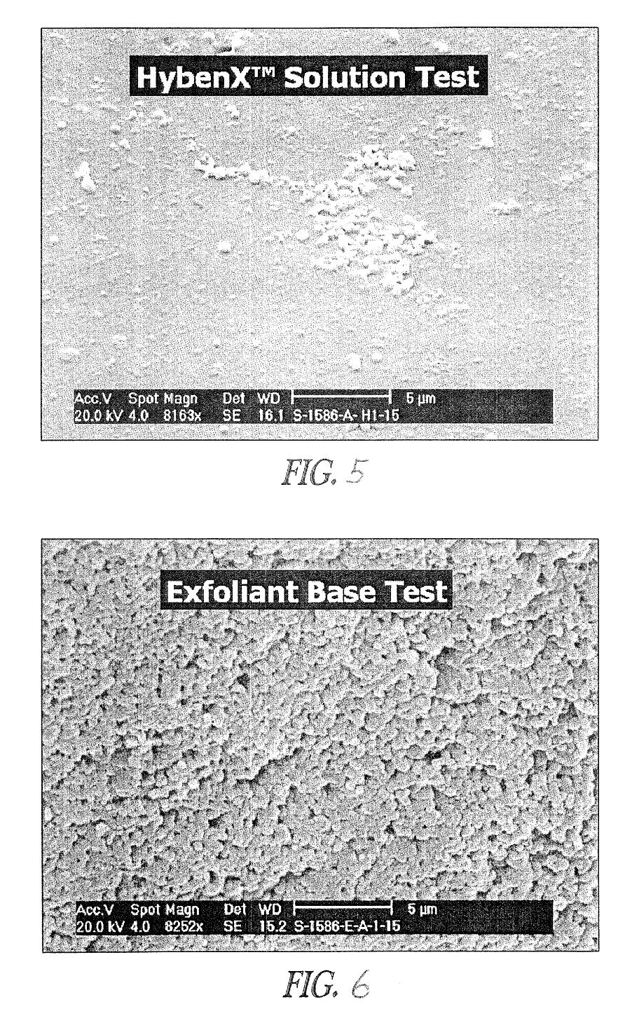

The purified phenolic compounds are shown in FIG. 2 and include phenol, guaiacol, salicylic acid, resorcinol and hydroquinone. The purified phenolics are treated with sulfuric acid to form sulfonic acids and sulfonate salts, as is shown in FIG. 1B. The sulfonate salts are treated with one or more of zinc, potassium and sodium to form a salt. Examples of other salts include alkali metal or alkaline earth metal salts and, particularly, calcium, magnesium, sodium, lithium, zinc, potassium, and iron salts. Phenolic compounds obtained from a variety of sources are usable to make the formulation described herein

Method and system embodiments described herein are usable to remove biofilm of virtually any microbe, including but not limited to bacterial, viral, prion, fungal, cyst, of plant origin, of animal origin. For some embodiments biofilms are of two types: nonspecific and specific. Nonspecific, reversible biofilm formation is mainly a physicochemical process. A number of classes of interaction are typically involved in nonspecific biofilm formation: Van der Waals forces, dipolar, electrostatic, hydrogen bonds, hydrophobic interactions. The number and strength of these interactions vary considerably from system to system, depending on the type of the surface and microbe involved. For instance, part or an entire external surface of some microbes is hydrophobic, and the biofilm formation of some bacterial strains onto hydrophobic sulphated polystyrene should correlate with bacterial hydrophobicity. The contact angle is a relative measure for representing the degree of hydrophobicity of a surface, which in most cases shows a correlation with the surface Gibbs energy (the surface Gibbs energy decreases with an increase in hydrophobicity).

Proteins such as lectins, which are carbohydrate-binding proteins, may serve as biofilms. Lectin-like, binding sites- mediated interactions have been considered important even for bacterial adhesion to inert surfaces such as hydroxyapatite.

Formulations that include the sulfonic acids of phenol and guaiacol are used to remove biofilms on virtually any surface, including skin or other human or other living being tissue and implantable medical devices. The term “biofilm” as used herein refers to substances that contain either single or multiple microbial species and that readily adhere to such diverse surfaces as river rocks, soil pipelines, teeth, mucous membranes, and medical implants. Biofilms are biological films that can develop and persist on solid substrates in contact with moisture, on soft tissue surfaces in living organisms and at liquid air interfaces. They can develop into structures several millimetres or centimeters in thickness and can cover large surface area.

Formulations that include the phenolsulfonate salts also have use as skin resurfacing products. A breakdown of these acid and salt products is shown in FIG. 2.

When phenol is treated with sulfuric acid, the reaction products include four isomers of phenolsulfonic acid. When guaiacol is treated with sulfuric acid, the reaction products include seven isomers, a mono- and a bis- form of guaiacolsulfonic acid. Other sources of phenolsulfonic acid include the four isomers. Other sources of guaical include the seven isomers, a mono- and a bis- form of guaiacolsulfonic acid.

Formulations prepared with Beechwood creosote are also usable for removing biofilms. One formulation embodiment, for removing biofilms is made from ingredients that include liquid phenol USP, guaiacol USP, sulfuric acid NF, purified water USP, and, for some embodiments FD&C Red No. 40. The formulation includes phenolsulfonic acid in a concentration of about 30%; guaiacolsulfonic acid in a concentration of about 32%; free sulfuric acid in a concentration of about 24%; water in a concentration of about 11.9% and FD&C Red No. 400 in a concentration of about 0.075%.

The odor of this formulation is mildly phenolic. The color is purple. The viscosity is about 1025 cPs at 25 degrees Centigrade. The total acidity is 9.1 mN/g. The density is about 1.59 g/mL. The application time is about 5 to 30 seconds. The formulation composition includes phenolsulfonic acid in a concentration of 30 percent by weight; guaiacolsulfonic acid in a concentration of 32 percent by weight; free sulfuric acid in a concentration of 24 percent by weight; water in a concentration of 11.9 percent by weight; and FD&C Red No. 40 in a concentration of 0.075 percent by weight. This formulation has a density of 1.59 g/mL, a total acidity of 9.1 mM/g, and a viscosity of 1025 cPs at 25 degrees Centigrade. The formulation does not include a tissue colorant.

Another formulation embodiment includes phenolsulfonic acid in a concentration of 25 to 80% by volume; guaiacolsulfonic acid in a concentration of 25 to 80% by volume; ammonium phenolsulfonate in a concentration of 0 to 5% by volume; free sulfuric acid in a concentration of 0 to 3% by volume; water in a concentration of 13 to 30% by volume and colorant in a concentration of 0.0075 to 0.020% by volume.

The formulation embodiments are applied to surfaces having a biofilm with an applicator, for some embodiments. The application contact time for treatment ranges from less than 1 to about 15 seconds for removal of at least about 80% of the biofilm from the surface. Effectiveness of the formulation embodiments described herein for removing biofilms are shown in FIGS. 8 to 12.

FIGS. 8A and 8D illustrate a low magnification of a P. aeruginosa PAOI culture, treated with a formulation embodiment of the invention, wherein after treatment, the culture is free of biofilm with a small amount of debris. FIG. 8B illustrates a low magnification colony on a surface of polyethylene glycol, peg, prior to treatment, clearly demonstrating the presence of a biofilm. FIG. 8C illustrates a very high magnification view of a biofilm colony of P. aeruginosa PAOI on the surface of peg, which has been treated with a formulation embodiment of the invention. Degenerated bacterial cells are shown. FIG. 8E illustrates a high magnification view of a biofilm colony on the surface of peg, which has been treated with a formulation embodiment of the invention. Degenerated bacterial cells are observable.

FIGS. 9A through 9D illustrate effectiveness of a formulation embodiment of the invention in destroying a P. aeruginosa PAOI biofilm from about 85% of a surface. Where biofilm is present, such as in FIGS. 9B, 9C and 9D, the P. aeruginosa PAOI biofilm appears as scattered microcolonies and contains degenerated cells.

FIGS. 10A, 10B, 10C and 10D illustrate a set of negative control cultures of P. aeruginosa PAOI on peg. These cultures have not been treated with any formulation of the invention. Approximately 95% of the surface is covered with bacterial biofilm. Biofilm bacteria wre healthy in appearance, i.e. rod shaped cells with extensive exopolysaccharide.

In another test, results of which are shown in FIGS. 11A, 11B and 11C, cultures of P. aeruginosa PAOI were treated with another formulation embodiment (Ex-base) for an exposure time of 15 seconds. Biofilm is present only on the peg tip and includes only degenerated cells.

A second set of negative controls are shown in FIGS. 12A, 12B, 12C, 12D, and 12E. These samples were exposed to saline for 15 seconds. About 100% of the surface is covered with bacterial biofilm of P. aeruginosa PAOI. Biofilm bacteria were healthy in appearance, with rod shaped cells having extensive exopolysaccharide.

Acceptable component ranges for formulation embodiments for removing biofilms from a surface, are as follows:

| Component | Concentration Ranges |
|---|---|
| Phenolsulfonic Acid | 25-80% by weight |
| Guaiacolsulfonic Acid | 25-80% by weight |
| Sulfosalicylic Acid | 0-30% by weight |
| Citric Acid | 0-30% by weight |
| Ammonium Phenolsulfonate | 0-32% by weight |
| Free Sulfuric Acid | 0-32% by weight |
| Water | 13-30% by weight |

The viscosity is 70 to 1000 cPs. The total acidity is 7.20 to 9.20 mM/g. The density is 1.46 to 1.59 g/mL. The application time is less than 1 second to not more than abut 15 seconds. The applicator includes in some embodiments, a vial, a pre-filled syringe, a swab, a patch or combinations of these applicators. The formulation optionally includes a colorant such as green or blue.

Devices for applying the formulations described herein are shown in FIG. 7 and include in one embodiment, a syringe and a cotton swab. In one embodiment, the syringe is prefilled. In one embodiment, formulation embodiments are enclosed within a swab device. In another embodiment, the formulation is enclosed in a container with a delivery mechanism that delivers the formulation drop-by-drop. In another embodiment, the formulation is metered. In one other embodiment, the formulation is enclosed in a container that includes a mechanism for heating or cooling the formulation. The mechanism includes chemicals, separated in compartments by a breakable seal, adjacent to the formulation embodiments described herein, that create an exothermic reaction when combined. In one embodiment, the chemicals are separated from the formulation by a wall. The chemicals are combined when a user breaks the breakable seal. The heating is transferred to the formulation through the wall. The packaged formulation embodiments are capable of being transported for sale to pharmacies and drug stores.

In another embodiment, formulation embodiments described herein are usable for sterilizing devices and components used in applications that include but are not limited to medical and dental applications. For some embodiments, formulations are passed through tubing to sterilize the tubing. For other embodiments, formulations are used to sterilize dental and medical equipment, such as catheters. Embodiments of the invention may be applied in forms that include gels, spray, lotion, shampoo, stick-based formulations, powder, cream, foam, ointment and structurant. For some embodiments, formulation embodiments are usable to remove biofilm from seeds. Formulation embodiments are also usable to remove biofilms that form along inner walls of conduits in industrial facilities and in household plumbing systems. Formulation embodiments are also useful to treat biofilms in cooling water systems used in power- generating plants, refineries, chemical plants, and air conditioning systems. Cooling water systems are often contaminated with airborne organisms entrained by air/water contact in cooling towers as well as waterborne organisms from the system's makeup water supply. Formulation embodiments are also usable to treat biofilms that form in water supply storage and conveying equipment.

Formulation and method embodiments are also usable to remove biofilms from locations that have implications in human and animal health Biofilms can present a serious threat to health as foci of chronic infections. For example, biofilm composed of Pseudomonas aeruginosa, the bacterium responsible for biofilm formed in the lungs of cystic fibrosis patients, is believed to be behind the fatal lung infections in patent suffering this disease. Biofilms have been implicated in periodontal disease, tooth decoy, prostate infections, kidney stones, tuberculosis. Legionnaire's disease and some infections of the middle ear.

Formulations and methods described herein are also usable to prevent infections resulting from medical intervention. For example, formulations and methods described herein can remove biofilms that form on medical devices including catheters, medical implants, dental equipment and contact lenses.

Formulations and method embodiments described herein are usable to prevent biofilm formation in bioimplants such as bone prosthesis, heart valves, pacemakers, stents, orthopaedic devices, ear implant devices, electrodes, dialysis devices and the like.

The biofilm treatment embodiments described herein have a benefit over antibiotic use in that organisms forming biofilm, ranging from prions to viruses, to bacteria to fungi, to cysts, and any other biofilm formers, do not develop a resistance to the biofilm formulations.

Formulations and systems described herein are believed to work by mechanisms of therapeutic action that include solvent/keratolytic; hygroscopic/dehydrating/ and denaturant/keratocoagulant. The solvent/keratolytic mechanixm penetrates and dissolves necrotic tissue. The hygroscopic/dehydrating mechanism reduces tissue edema. Solvation of the formulation is exothermic and releases an acidification mechanism. The denaturant/keratocoagulant mechanism produces acidification necrosis and oxidation and surrogate eschar/clot which produces a protective natural bandage.

System and formulation embodiments described herein impart virtually instantaneous pain relief; accelerated ulcer healing; an infectious organism kill; and effectiveness in a one time treatment. The system and formulation embodiments act by a self-limited action that is not harmful to healthy mucosa. The system and formulation form a barrier membrane and neutralize acid.

Since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes, which come within the meaning and range of equivalency of the claims, are intended to be embraced therein.

1. A method for removing a biofilm from a surface, comprising exposing the biofilm to a formulation comprising sulfonic acid derivatives of hydroxybenzenes and hydroxymethoxybenzene.

2. The method of claim 1, wherein the biofilm is for at least about fifteen seconds.

3. The method of claim 1 wherein the formulation is obtained from beechwood creosote.

4. A system for removing a biofilm from a surface comprising a formulation comprising phenolsulfonic acid guaiacolsulfonic acid, sulfosalicylic acid, citric acid, and ammonium phenolsulfonate; and an applicator for applying the formulation to a biofilm for at least about 15 seconds.

5. The method of claim 1 wherein the surface is living.

6. The method of claim 1, wherein the surface is not living.

7. The system of claim 4, wherein the applicator comprises a spray.

8. The system of claim 4, wherein the applicator comprises a swab or a wipe.

9. The system of claim 4, wherein the formulation is as one or more of a gel, spray, lotion, shampoo, stick-based formulation, powder, cream, foam, ointment or structurant.

10. The method of claim 1, wherein the sulfonic acid derivatives of hydroxybenzenes and hydroxymethoxybenzene comprise purified phenolics.

11. The method of claim 1, wherein the sulfonic acid derivatives comprise at least one of four isomers of phenolsulfonic acid.

12. The method of claim 1, wherein the sulfonic acid derivatives comprise at least one of seven isomers of guaiacolsulfonic acid.

13. The method of claim 1, wherein the sulfonic acid derivative comprises ammonium phenolsulfonate.

14. The method of claim 1, wherein the sulfonic acid derivative comprises potassium guaiacolsulfonate.

15. A system for making a formulation for removing a spedific type of biofilm from a surface comprising:
receptacles for containing two or more ingredients of phenolsulfonic acid, guaiacolsulfonic acid, sulfosalicylic acid, ammonium phenolsulfonate, potassium guaiacolsulfonate; and
a mechanism calibrated to mix the ingredients together to make a formulation for treating the specific type of biofilm.

16. A method for removing biofilm from seeds comprising exposing the seeds to a formulation comprising sulfonic acid derivatives of hydroxybenzenes and hydroxymethoxybenzene.

17. A method for treating a biofilm without stimulating a resistance in a living being, comprising: exposing the biofilm to a formulation comprising sulfonic acid derivatives of hydroxybenzenes and hydroxymethoxybenzene.

18. The method of claim 17, wherein the living being is human.

19. The method of claim 17, wherein the living being is non-human.
